(19) United States
(12) Patent Application Publication
(10) Pub. No.: US 2003/0181941 A1
(43) Pub. Date: Sep. 25, 2003

(54) Nasal dilator for oxygen enhancement

(76) Inventors: Arthur T. Bruggisser, Dania Beach, FL (US); Arthur W. Bruggisser, Dania Beach, FL (US)

Correspondence Address: David P. Lhota, Stearns Weaver Miller, Suite 1900, 200 East Broward Boulevard, Fort Lauderdale, FL, 33301, US

(21) Appl. No.: 10/392,050

(22) Filed: Mar. 19, 2003

Related U.S. Application Data
(60) Provisional application No. 60/365,613, filed on Mar. 19, 2002.

(51) Int. Cl. A61M 29/00

(52) U.S. Cl. 606/196

(57) ABSTRACT

A nasal dilator that is insertable and self-supportable in a person's nose over the septum and opens the nasal passages by physical action against the nostrils from within the nose, wherein the nasal dilator comprises a pair of exterior arms each having a paddle shaped wing or lip and centrally located ball-shaped protrusions and a center clasp that engages and secures to the septum of a person's nose. The dilator is made from a soft plastic-like or rubber-like material that is washable and flexible for inserting, securing and removing the dilator and for universal use.

### CROSS REFERENCE TO RELATED APPLICATIONS

This application claims the benefit of provisional application Serial No. 60/365,613 filed Mar. 19, 2002.

### STATEMENT REGARDING FEDERALLY SPONSORED RESEARCH OR DEVELOPMENT

N/A

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office patent file or records, but otherwise reserves all copyrights rights whatsoever.

### BACKGROUND OF THE INVENTION

1. Field of the Invention

This invention relates generally to a breathing enhancer, and more particularly, to a nasal dilator that is removably insertable in a person's nose to further open the nasal passages for enhancing the intake of oxygen.

2. Description of the Background Art

The ability to breath freely is obviously vital to life and can enhance physical activity. Increasing the amount of oxygen intake can help enhance the physical performance of athletes, laborer's and person's that experience difficulty in breathing, such as people that suffer from apnea. Widening the nasal passages enhances oxygen intake. Prior art devices, such as tape, are placed on the outside of the nose to pull the nostrils upward to open the nasal passages. Prior art devices, however, are unreliable and limited in effectiveness and typically fall off when exposed to moisture, such as sweat. A device that enhanced breathing while addressing the shortcomings of the prior art would be well received. The instant invention addresses this unfulfilled need in the prior art.

### BRIEF SUMMARY OF THE INVENTION

In light of the foregoing, it is a primary object of the instant invention to provide a nasal dilator device that comfortably engages a person's nose and opens the nasal passages to enhance breathing without the use of drugs.

It is also an object of the instant invention to provide a nasal dilator that enhances breathing during exercise, work and sleep.

It is an additional object of the instant invention to provide a nasal dilator that is flexible and washable.

It is a further object of the instant invention to provide a nasal dilator that is impervious to water and does not retain tastes or odors when washed.

It is another object of the instant invention to provide a nasal dilator that opens the nasal passages from inside the nostrils.

It is also an object of the instant invention to provide a nasal dilator that may be securely mounted to a person's septum.

Based on the foregoing, the instant invention provides a nasal dilator that is insertable and self-supportable in a person's nose over the septum and opens the nasal passages by physical action without the use of drugs. The nasal dilator facilitates the opening of the nasal passages during exercise, work and sleep to facilitate greater airflow, and therefore more oxygen, into the nostrils during sleep, exercise, work and sexual activity. The nasal dilator comprises a flare mechanism and support mechanism joined by a common base. The flare mechanism comprises a pair of exterior arms each having a paddle shaped wing or lip and centrally located ball-shaped protrusions. The wings/lips are flexible and exert an outward force against the nostrils when inserted in a person's nose. The support mechanism comprises a center clasp that engages and secures to the septum of a person's nose without the aid of additional securing mechanisms. The exterior arms and clasp extend from a common base member. The dilator is preferably made from a soft plastic-like or rubber-like material that is washable and flexible for virtually universal use.

In accordance with these and other objects, which will become apparent hereinafter, the instant invention will now be described with particular reference to the accompanying drawings.

### BRIEF DESCRIPTION OF THE SEVERAL VIEWS OF THE DRAWINGS

FIG. 1 is an illustrative view of the preferred embodiment of the instant invention inserted in a person's nasal passages.

FIG. 2 is an elevational view of the preferred embodiment of the instant of the instant invention in a post insertion position.

FIG. 3 is an elevational view of the preferred embodiment of the instant of the instant invention prior to insertion into the nasal passages.

### DETAILED DESCRIPTION OF THE INVENTION

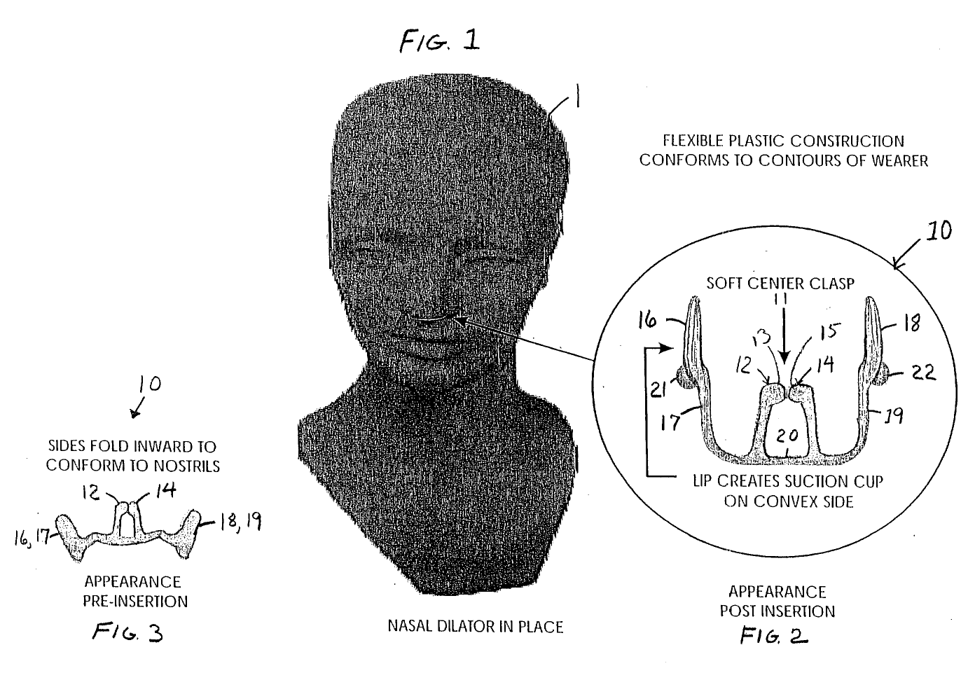

With reference to the drawings, FIGS. 1-3 depict the preferred and alternative embodiments of the instant invention which is generally referenced as a nasal dilator and, or by numeric character 10. The nasal dilator 10 comprises a reusable device that is insertable and self-supportable in a person's 1 nose over the septum and opens the nasal passages by physical action without the use of drugs. The nasal dilator 10 allows the user 1 to open and keep open the nasal passages during exercise, work and sleep. The nasal dilator 10 opens the nasal passages to facilitate greater airflow, and therefore more oxygen, into the nostrils during sleep, exercise, work and sexual activity.

With reference to FIG. 2, the nasal dilator 10 comprises a flare mechanism and support mechanism joined by a common base. The flare mechanism comprises a first exterior arm 17, second exterior arm 19, first paddle shaped wing/lip 16, second paddle shaped wing/lip 18, first ball-shaped protrusion 21, and second ball-shaped protrusion 21. The first paddle shaped wing 16 and second paddle shaped wing 18 are defined by the upper portion or half of the first and second exterior arms 17 and 19, respectively. The center clasp 11 comprises a first inner arm 12 and second inner arm 14. The first and second exterior arms 17, 19 and first and second interior arms 12, 14 protrude from a common base member 20.

The nasal dilator 10 preferably comprises an integrally formed, unitary structure preferably manufactured from a soft, flexible, washable and plastic, plastic-like, rubber or rubber-like material. The first and second exterior arms 17, 19 extend upward from opposite ends of the base member 20 and are terminated by the first and second paddle shaped wings 16, 18, respectively. The base member 20 preferably blends into the first and second exterior arms 17, 19. The wings 16, 18 are slightly concave and turned outward to facilitate proper and secure placement in the nasal passages and force against the nostrils. The concave design also facilitates the creation of a suction force on the convex side to enhance secure placement. The wings 16, 17 can be folded inward, as shown in FIG. 3, to conform to the nostrils. A small ball shaped protrusion 21, 22 is molded into the bottom side of each of the paddle shaped wings 16, 18 on the side of the recess proximal the upper end of the extensions 17, 19. The protrusions 21, 22 add structural integrity and accommodate the bending of the wings 16, 18.

With reference to FIG. 2, a soft center clasp 11 projects upward from the center of the base member 20. The center clasp 11 comprises two short and substantially perpendicularly oriented arms or clasps 12, 14 terminated with substantially ball shaped ends. The first and second clasps 12, 14 are preferably angled inward toward the other, such that the free ends of the clasps 12, 14 touch or virtually touch. The clasps' 12, 14 have ball shaped ends 13, 15, respectively, that touch or come close to contact when the device 10 is not inserted into the nose. The first and second clasps 12, 14 preferably comprise a flexible plastic or plastic-like material having a biasing property that facilitates the storage of energy when urged apart such that the clasps 12, 14 move inward to seek their resting position when released. The clasps 12, 14 are resilient and can be pulled apart prior to insertion. When the nasal dilator 10 is inserted, the ball shaped ends 13, 15 engage or grasp the septum to secure the nasal dilator 10 on the septum. In one embodiment, the length of the clasps 12, 14 are approximately equal to the spacing between them. The clasps 12, 14 results in a substantially “U” shaped section with the base member 20 and define an adjustable width that fits over the septum as the device is inserted into the nose.

The nasal dilator 10 is preferably made from molded silicone, polyurethane or other non-allergenic flexible soft plastic or rubber. The nasal dilator 10 may be made from colored or colorless, opaque or transparent plastic or plastic-like material. The size of the ball shaped protrusions 13, 15 may be made in several sizes for increased comfort. The spacing between the wings 16, 18 may be made in several different sizes to accommodate nasal openings of different persons 1. The base member 20, exterior arms 17, 19 and inner arms 12, 14 preferably have a substantially circular or curved cross section for comfort and flexibility.

The nasal dilator 10 is washed with soap and water to remove foreign substances. The device's 10 wings 16, 18 are inserted into the nostrils with the ball shaped protrusions 21, 22 facing outward. The septum clasps 12, 14 are placed in the nasal passages on each side of the septum. The device is adjusted for a comfortable fit and proper airflow. By using the nasal dilator 10, the user should experience an increased flow of air through the nostrils. The device 10 can be removed quickly and easily, washed and stored for future use.

The instant invention has been shown and described herein in what is considered to be the most practical and preferred embodiment. It is recognized, however, that departures may be made therefrom within the scope of the invention and that obvious structural and/or functional modifications will occur to a person skilled in the art.

What is claimed is:

1. A nasal dilator device adapted for insertion in a person's nasal passageways over the septum, said device comprising:
a base member;
flare means, extending from said base member, for exerting an outward force on the nostrils from inside a person's nose; and
support means, extending upward from said base member between said flare means, for engaging a person's septum.

2. A device as recited in claim 1, wherein said dilator is flexible and water resistant.

3. A device as recited in claim 1, wherein said flare means extends outward from opposite ends of the base member.

4. A device as recited in claim 1, wherein said flare means stores energy when urged from its rested position and exerts an outward force when released to exert a force on the inner walls of a person's nostrils.

5. A device as recited in claim 1, wherein said support means extends outward from opposite ends of the base member.

6. A device as recited in claim 1, wherein said support means stores energy when urged from its rested position and exerts an inward force when released to exert a force on the person's septum.

7. A device as recited in claim 1, wherein said flare means comprises:
a pair of flexible, semi-rigid arms extending upward from opposite ends of said base member.

8. A device as recited in claim 7, wherein said flare means comprises:
at least one lip defined by each of said arms, said lips being flexible for bending and releasing inside the nostrils to exert an outward force.

9. A device as recited in claim 8, wherein said flare means comprises:
at least one ball-shaped protrusion defined at the base of said at least one lip.

10. A device as recited in claim 8, wherein said at least one lip is at least partially concave for facilitating a suction force against the nostrils.

11. A device as recited in claim 1, wherein said support means comprises:
a pair of flexible, semi-rigid septum engaging arms extending upward and at least partially inward toward each other from a mid-section of said base member, said arms being spaced apart a distance that facilitates a clasping force on a person's septum.

12. A device as recited in claim 11, wherein said support means comprises:
at least one ball-shaped protrusion defined proximal a free end of each of said septum engaging arms, said ball-shaped protrusions being urged toward each other when said arms are spread apart.

13. A device as recited in claim 11, wherein said septum engaging arms each have a length approximately equal to the spacing between said septum engaging arms.

14. A device as recited in claim 1, wherein said device comprises a flexible, semi-rigid material having memory for returning to a resting position when manipulated, said material having properties that repel water to avoid water absorption.

15. A device as recited in claim 1, wherein said device has a predetermined color.

16. A device as recited in claim 1, wherein said device is at least partially opaque.

17. A device as recited in claim 8, wherein said at least one lip is at least partially paddle shaped for adapting to the interior walls of a person's nostrils.
